(19) United States
(12) Patent Application Publication
(10) Pub. No.: US 2010/0003676 A1
(43) Pub. Date: Jan. 7, 2010

(54) THERMOSTABLE DNA POLYMERASE OF THE ARCHAEAL AMPULLAVIRUS ABV AND ITS APPLICATIONS

(75) Inventors: Xu Peng, Gentofte (DK); Monika Haering, Altoetting (DE); Roger Garrett, Humlebaek (DK); David Prangishvili, Versailles (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: UNIVERSITY OF COPENHAGEN, Copenhagen K (DK)

(21) Appl. No.: 12/300,377

(22) PCT Filed: May 14, 2007
(86) PCT No.: PCT/IB07/02319

§ 371 Date: Nov. 11, 2008

(30) Foreign Application Priority Data
May 12, 2006 (EP) 06290764.7

(51) Int. Cl. C12Q 1/68; C12N 9/12; C12N 15/54; C12N 15/74; C12N 1/21; C12P 19/34

(52) U.S. Cl. 435/6; 435/194; 536/23.2; 435/320.1; 435/252.3; 435/91.5; 435/91.2; 435/91.51

(57) ABSTRACT

The present invention is directed to the thermostable DNA-polymerase protein of the archaeal ampullavirus ABV (Acidianus Bottle-shaped virus) and the nucleic acid encoding said DNA polymerase. The invention also relates to method of synthesizing, amplifying or sequencing nucleic acid implementing said DNA polymerase protein and kit or apparatus comprising said DNA polymerase protein.

The present invention is directed to the thermostable DNA polymerase protein of the archaeal ampullavirus ABV (Acidianus Bottle-shaped virus) and the nucleic acid encoding said DNA polymerase. The invention also relates to method of synthesizing, amplifying or sequencing nucleic acid implementing said DNA polymerase protein and kit or apparatus comprising said DNA polymerase protein.

The double-stranded (ds) DNA viruses of hyperthermophilic Crenarchaeota exhibit remarkably diverse morphotypes and genome structures and, on the basis of these properties several have already been assigned to six new viral families: spindle-shaped Fuselloviridae, filamentous Lipothrixviridae, rod-shaped Rudiviridae, droplet-shaped Guttaviridae, spherical Globuloviridae and two-tailed Bicaudaviridae (reviewed in Prangishvili et al., 2001; Prangishvili and Garrett, 2004, 2005). A novel virus was recently discovered which exhibited a unique bottle-shaped morphology and it was tentatively assigned to a new family, the Ampullaviridae (Häring et al., 2005a).

A variety of nucleic acid amplification techniques, developed as tools for nucleic acid analysis and manipulation, have been successfully applied for clinical diagnosis of genetic and infectious diseases. Amplification techniques can be grouped into those requiring temperature cycling (PCR and ligase chain reaction) and isothermal systems (amplification systems (3SR and NASBA), strand-displacement amplification, and Qβ replication systems). Two aspects are frequent caveats in these procedures: fidelity of synthesis and length of the amplified product.

Development of an amplification system relying on the mechanism of phage phi29 (φ29) DNA replication has been the object of publications and patent documents (Dean et al., Genome Res. June 2001;11(6):1095-9; Mendez et al., EMBO J., 1997, 1;16(9):2519-27; Hutchison et al., Proc Natl Acad Sci USA., 2005, 102(48):17332-6; Mamone, Innovations Forum: GenomiPhi DNA amplification, Life Sciences News 14, 2003 Amersham Biosciences; Blanco et al., 1994; EP 0 862 656 or U.S. Pat. No. 5,001,050).

The phi29 DNA polymerase is a highly processive polymerase featuring strong strand displacement activity which allows for highly efficient isothermal DNA amplification (Blanco et al., Proc. Natl. Acad. Sci. USA, 81, 5325-5329, 1984 and J. Biol. Chem., 264, 8935-8940, 1989). The phi29 DNA Polymerase also possesses a 3′=>5′ exonuclease (proofreading) activity acting preferentially on single-stranded DNA (Garmendia J. Biol. Chem., 267, 2594-2599, 1992).

Among its features, we can cited its highest processivity and strand displacement activity among known DNA polymerases—more than 70 kb long DNA stretches can be synthesized (Blanco et al., 1989), its highly accurate DNA synthesis (Esteban et al., J. Biol. Chem., 268, 4, 2719-2726, 1993), its high yields of amplified DNA even from minute amounts of template and the amplification products can be directly used in downstream applications (PCR, restriction digestion, SNP genotyping, etc.). Numerous specific applications were developed implementing this particular DNA polymerase such as Rolling Circle Amplification (RCA) (Lizardi et al., Nat. Genet., 19, 225-232, 1998; Dean et al., Genome Res., 11, 1095-1099, 2001; Baner et al., Nucleic Acids Res., 26, 5073-5078, 1998). Multiple Displacement amplification (MDA) (Dean et al., Proc. Natl. Acad. Sci. USA, 99, 5261-5266, 2002), unbiased amplification of whole genome or DNA template preparation for sequencing. This system would be adequate for faithful amplification of DNA molecules longer than 70 kb (Blanco et al., 1989), largely over the size limit obtained with the amplification systems available to date. This procedure of isothermal TP-primed amplification (“TP” for terminal protein) would exploit the particular properties of phi29 DNA polymerase: (1) ability to use a protein as primer, (ii) intrinsic high processivity (>70 kb), and (ii) strand displacement coupled to DNA synthesis. The specific activity for this phi29 DNA polymerase is given for a temperature of 30° C. and it is precised that this phi29 DNA polymerase is inactivated at 65° C.

Currently there is a need for a new DNA polymerase belonging to the protein-primed DNA polymerase family such as phi29 DNA polymerase which can work at temperature significantly superior to 30° C. and which is not completely inactivated at 60° C.

This is the object of the present invention.

After sequencing and annoted the complete genomic sequence of the virus ABV (Acidianus Bottle-shaped virus) infecting hyperthermophilic archaea of the genera Acidianus, the inventors have demonstrated a nucleic sequence encoding a DNA-dependent DNA polymerase. Surprisingly, the analysis of the protein sequence indicated that it belongs to the protein-primed DNA polymerase family. The gene for DNA polymerase was heterologously expressed in E. coli and DNA polymerization activity of the recombinant protein has been confirmed. This novel enzyme, similar to known viral DNA polymerases, is highyly processive and self sufficient, not requiring auxiliary proteins. Due to these features the enzyme can have significant advantages as a tool for DNA amplification by PCR. Being protein-primed thermostable viral enzyme it can be much more efficient in exponential amplification of single- or double-stranded linear DNA (i.e. by the GenomiPhi procedure developed by Amersham) than bacteriophage Phi29 DNA polymerase, a mesophilic protein-primed enzyme, currently utilized in this procedure. GenomiPhi Amplification Kit of Amersham enables to perform unlimited DNA tests from a small number of cells or limited amount of precious sample and is an easy genomic DNA amplification method that representatively amplifies the whole genome.

So, in a first aspect, the present invention is directed to an isolated DNA polymerase selected from the group of polypeptides consisting of:
a) the polypeptide having the amino acid sequence of SEQ ID NO: 1;
b) a fragment of a) having a DNA polymerase activity,
c) a chimeric polypeptide comprising at least the SEQ ID NO: 1 fragments allowing the DNA polymerase activity of said DNA polymerase of a);
d) a polypeptide having the amino acid sequence of SEQ ID NO: 1 wherein the exonuclease sites Exo I, Exo II and/or Exo III as identified in FIG. 4 have been mutated or deleted to result in a DNA polymerase polypeptide having a significantly less or no detectable exonuclease activity compared to the polypeptide having the amino acid sequence of SEQ ID NO: 1;
e) a polypeptide having sequence which is at least 80% identity after optimum alignment with the sequence SEQ ID NO: 1, or as defined in b) to d), said polypeptide having a DNA polymerase activity, preferably at a temperature of 50° C. or superior to 50° C.

In a preferred embodiment, the fragment having a DNA polymerase activity has at least 50, 100, 150, 200, 250, 300, 350, 400, 450, 500 or 600 amino acids.

In a preferred embodiment, the DNA polymerase according to the invention is isolated from ABV or from the ABV gene encoding the DBA polymerase.

In a more preferred embodiment, the DNA polymerase of the present invention comprises at least the Pol I, Pol IIa, Pol IIb, Pol III and Pol IV fragments of SEQ ID NO: 1 as identified in FIG. 4.

Referring to the FIG. 4, the polypeptide of the present invention having its DNA polymerase preserved but a deficient or significantly less exonuclease activity than the polypeptide having the sequence SEQ ID NO: 1 can be selected by taking into account the amino acid sequence homology with other polymerases and those mutations known to reduce exonuclease activity of DNA polymerase (Derbyshire et al., Science, Apr. 8, 1988; 240(4849):199-201). Generally, the amino acid at these portions shown as Exo I, Exo II and/or Exo III in FIG. 4 can be either deleted or replaced with different amino acids. Large deletions or multiple replacement of amino acids at these Exo I, Exo II and/or Exo III positions can be also carried out. After mutagenesis the polypeptide having the sequence SEQ ID NO: 1, the level of exonuclease activity is measured and the amount of DNA polymerase activity determined to ensure it is sufficient for use in the present invention.

The term “5′ exonuclease activity” refers to the presence of an activity in a protein which is capable of removing nucleotides from the 5′ end of an oligonucleotide. 5′ exonuclease activity may be measured using any of the assays provided herein.

The DNA polymerases of this invention include polypeptides which have been genetically modified to reduce the exonuclease activity of that polymerase, as well as those which are substantially identical (identity to at least 80%) naturally-occurring ABV DNA polymerase or a modified polymerase thereof, or to the equivalent enzymes enumerated above. Each of these enzymes can be modified to have properties similar to those of the ABV DNA polymerase. It is possible to isolate the enzyme from ABV virus infected cells directly, but preferably the enzyme is isolated from cells which over-produce it (recombinant expression).

The term “exonuclease activity” refers to the presence of an activity in a protein which is capable of removing nucleotides from the 3′ end or from the 5′ end of an oligonucleotide. Such exonuclease activity may be measured using any of the exonuclease activity assays well known by the skilled person.

The term “DNA polymerase activity” refers to the ability of an enzymatic polypeptide to synthesize new DNA strands by the incorporation of deoxynucleoside triphosphates. The example 4 below provides an example of assay for the measurement of DNA polymerase activity. Such DNA polymerase activity may be measured using any of the DNA polymerase activity assays well known by the skilled person. A protein which can direct the synthesis of new DNA strands (DNA synthesis) by the incorporation of deoxynucleoside triphosphates in a template-dependent manner is said to be “capable of DNA polymerase activity”.

In the present description, the terms polypeptides, polypeptide sequences, peptides and proteins are interchangeable.

The terms “identical” or percent “identity”, in the context of two or more polypeptide sequences, refer to two or more sequences or subsequences that are the same or have a specified percentage of amino acid residues that are the same (i.e., about 80% identity, preferably 85%, 90%, 95%, 98%, 99%, or higher identity over a specified region when compared and aligned for maximum correspondence over a comparison window or designated region) as measured using a BLAST or BLAST 2.0 sequence comparison algorithms with default parameters, or by manual alignment and visual inspection (see, e.g., NCBI web site). The definition also includes sequences that have deletions and/or additions, as well as those that have substitutions. As described below, the preferred algorithms can account for gaps and the like. Preferably, identity exists over a region that is at least about 25 amino acids in length, or more preferably over a region that is 25-75 amino acids in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

Methods of alignment of sequences for comparison are well-known in the art. A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990).

For example, it is possible to use the BLAST program, “BLAST 2 sequences” (Tatusova et al., “Blast 2 sequences—a new tool for comparing protein and nucleotide sequences”, FEMS Microbiol Lett. 174:247-250) available on the site http://www.ncbi.nlm.nih.gov/gorf/b12.html, the parameters used being those given by default (in particular for the parameters “open gap penalty”: 5, and “extension gap penalty”: 2; the matrix chosen being, for example, the matrix “BLOSUM 62” proposed by the program), the percentage of identity between the two sequences to be compared being calculated directly by the program.

By amino acid sequence having at least 80%, preferably 85%, 90%, 95%, 98%, 99%, or higher identity with a reference amino acid sequence, those having, with respect to the reference sequence, certain modifications, in particular a deletion, addition or substitution of at least one amino acid, a truncation or an elongation are preferred. In the case of a substitution of one or more consecutive or nonconsecutive amino acid(s), the substitutions are preferred in which the substituted amino acids are replaced by “equivalent” amino acids. The expression “equivalent amino acids” is aimed here at indicating any amino acid capable of being substituted with one of the amino acids of the base structure without, however, essentially modifying the DNA polymerase activity of the reference polypeptide and such as will be defined later, especially in the example 4, last paragraph.

These equivalent amino acids can be determined either by relying on their structural homology with the amino acids which they replace, or on results of comparative trials of DNA polymerase activity between the different polypeptides capable of being carried out. By way of example, mention is made of the possibilities of substitution capable of being carried out without resulting in a profound modification of the DNA polymerase activity of the corresponding modified polypeptide. It is thus possible to replace leucine by valine or isoleucine, aspartic acid by glutamic acid, glutamine by asparagine, arginine by lysine, etc., the reverse substitutions being naturally envisageable under the same conditions.

So, in a second aspect, the present invention provides a nucleic acid encoding a DNA polymerase polypeptide according to the invention, particularly the nucleic acid having the sequence SEQ ID NO: 2 or having a sequence which is at least 80% identity after optimum alignment with the sequence SEQ ID NO: 2, the polypeptide encoded by said nucleic acid having a DNA polymerase activity, preferably at a temperature of 50° C. or superior to 50° C.

In the present description, the terms nucleic acid, polynucleotide, oligonucleotide, or acid nucleic or nucleotide sequence are interchangeable.

In another aspect, the invention encompasses a vector, preferably a cloning or an expression vector, comprising the nucleic acid of the invention.

In a preferred embodiment, the vector according to the invention is characterized in that said nucleic acid is operably linked to a promoter.

The invention aims especially at cloning and/or expression vectors which contain a nucleotide sequence according to the invention.

The vectors according to the invention preferably contain elements which allow the expression and/or the secretion of the nucleotide sequences in a determined host cell. The vector must therefore contain a promoter, signals of initiation and termination of translation, as well as appropriate regions of regulation of transcription. It must be able to be maintained in a stable manner in the host cell and can optionally have particular signals which specify the secretion of the translated protein. These different elements are chosen and optimized by the person skilled in the art as a function of the host cell used. To this effect, the nucleotide sequences according to the invention can be inserted into autonomous replication vectors in the chosen host, or be integrative vectors of the chosen host.

Such vectors are prepared by methods currently used by the person skilled in the art, and the resulting clones can be introduced into an appropriate host by standard methods, such as lipofection, electroporation, thermal shock, or chemical methods.

The vectors according to the invention are, for example, vectors of plasmidic or viral origin. They are useful for transforming host cells in order to clone or to express the nucleotide sequences according to the invention.

In a preferred embodiment, the vector of the present invention is the plasmidic vector contained in the bacteria which has been deposited according to the Budapest Treaty at the C.N.C.M. (Collection Nationale de Cultures de Microorganismes, Institut Pasteur, Paris, France) the 28 Apr. 2006 under the number I-3601.

This cloned plasmidic vector is the vector pET30a wherein the nucleic sequence of the DNA polymerase of the invention has been inserted between the NdeI and XbaI sites of the pET30a plasmid.

The term “expression vector” refers to a recombinant DNA molecule containing the desired coding nucleic acid sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

In another aspect, the present invention relates to a host cell comprising the vector according to the invention, particularly the recombinant bacteria which has been deposited according to the Budapest Treaty at the C.N.C.M. (Collection Nationale de Cultures de Microorganismes, Institut Pasteur, Paris, France) the 28 Apr. 2006 under the number I-3601.

The DNA polymerase polypeptide of the present invention may be expressed in either prokaryotic or eukaryotic host cells. Nucleic acid encoding the DNA polymerase polypeptide of the present invention may be introduced into bacterial host cells by a number of means including transformation of bacterial cells made competent for transformation by treatment with calcium chloride or by electroporation. If the DNA polymerase polypeptide of the present invention are to be expressed in eukaryotic host cells, nucleic acid encoding the DNA polymerase polypeptide of the present invention may be introduced into eukaryotic host cells by a number of means including calcium phosphate co-precipitation, spheroplast fusion, electroporation and the like. When the eukaryotic host cell is a yeast cell, transformation may be affected by treatment of the host cells with lithium acetate or by electroporation or any other method known in the art. It is contemplated that any host cell will be useful in producing the peptides or proteins or fragments thereof of the invention.

The cells transformed according to the invention can be used in processes for preparation of recombinant polypeptides according to the invention. The processes for preparation of a polypeptide according to the invention in recombinant form, characterized in that they employ a vector and/or a cell transformed by a vector according to the invention, are themselves comprised in the present invention.

Preferably, a cell transformed by a vector according to the invention is cultured under conditions which allow the expression of said polypeptide and said recombinant peptide is recovered.

In another aspect, the present invention relates to a method of producing a DNA polymerase, said method comprising:
(a) culturing the host cell according to the invention in conditions suitable for the expression of said nucleic acid; and
(b) isolating said DNA polymerase from said host cell.

Said host cell can be a prokaryotic or an eukaryotic cell.

As has been said, the host cell can be chosen from prokaryotic or eukaryotic systems. In particular, it is possible to use nucleotide sequences facilitating secretion in such a prokaryotic or eukaryotic system. A vector according to the invention carrying such a sequence can therefore advantageously be used for the production of recombinant proteins, intended to be secreted. In effect, the purification of these recombinant proteins of interest will be facilitated by the fact that they are present in the supernatant of the cell culture rather than in the interior of the host cells.

In another aspect, the present invention encompasses a method of synthesizing a double-stranded DNA molecule comprising:
(a) hybridizing a primer to a first DNA molecule; and
(b) incubating said DNA molecule of step (a) in the presence of one or more deoxyribonucleoside triphosphates or analogs thereof and the polypeptide according to the invention, under conditions sufficient to synthesize a second DNA molecule complementary to all or a portion of said first DNA molecule.

In another aspect, the present invention encompasses a method of synthesizing a single-stranded DNA molecule comprising:
(a) the synthesis of a double-stranded DNA molecule by a method according to the invention; and
(b) denaturing the double-stranded DNA molecule obtained in step (a); and
(c) recovering the single-stranded DNA molecule obtained in step (b).

In another aspect, the present invention encompasses a method for production of DNA molecules of greater than 10 kilobases in length comprising the method according to the invention, wherein the first DNA molecule: which serve as a template in step (a) is greater than 10 kilobases.

In the method according to the invention, said deoxyribonucleoside triphosphates are selected from the group consisting of dATP, dCTP, dGTP and dTTP.

In another aspect, the present invention encompasses a method for amplifying a double stranded DNA molecule, comprising:
(a) providing a first and second primer, wherein said first primer is complementary to a sequence at or near the 3′-termini of the first strand of said DNA molecule and said second primer is complementary to a sequence at or near the 3′-termini of the second strand of said DNA molecule;
(b) hybridizing said first primer to said first strand and said second primer to said second strand in the presence of the polypeptide according to the invention, under conditions such that a nucleic acid complementary to said first strand and a nucleic acid complementary to said second strand are synthesized;
(c) denaturing
said first and its complementary strands; and
said second and its complementary strands; and
(d) repeating steps (a) to (c) one or more times.

It is also preferred that the step of amplifying is performed by PCR, or PCR-like method, or RT-PCR reaction implementing the polypeptide having a DNA polymerase activity (DNA polymerase polypeptide).

“PCR” describes a method of gene amplification which involves sequenced-based hybridization of primers to specific genes within a DNA sample and subsequent amplification involving multiple rounds of annealing (hybridization), elongation and denaturation using a heat-stable DNA polymerase.

“RT-PCR” is an abbreviation for reverse transcriptase-polymerase chain reaction. Subjecting mRNA to the reverse transcriptase enzyme results in the production of cDNA which is complementary to the base sequences of the MRNA. Large amounts of selected cDNA can then be produced by means of the polymerase chain reaction which relies on the action of heat-stable DNA polymerase.

“PCR-like” will be understood to mean all methods using direct or indirect reproductions of nucleic acid sequences, or alternatively in which the labeling systems have been amplified, these techniques are of course known, in general they involve the amplification of DNA by a polymerase; when the original sample is an RNA, it is advisable to carry out a reverse transcription beforehand. There are currently a great number of methods allowing this amplification, for example the so-called NASBA “Nucleic Acid Sequence Based Amplification”, TAS “Transcription based Amplification System”, LCR “Ligase Chain Reaction”, “Endo Run Amplification” (ERA), “Cycling Probe Reaction” (CPR), and SDA “Strand Displacement Amplification”, methods well known to persons skilled in the art.

When using MRNA, the method may be carried out by converting the isolated mRNA to cDNA according to standard methods using reverse transcriptase (RT-PCR).

In another aspect, the present invention encompasses a method of preparing cDNA from mRNA, comprising:
(a) contacting MRNA with an oligo(dt) primer or other complementary primer to form a hybrid, and
(b) contacting said hybrid formed in step (a) with the DNA polymerase polypeptide according to the invention and DATP, dCTP, dGTP and dTrP, whereby a cDNA-RNA hybrid is obtained.

The present invention is further directed to a method of preparing dsDNA (double strand DNA) from mRNA, comprising:
(a) contacting mRNA with an oligo (dT) primer or other complementary primer to form a hybrid; and
(b) contacting said hybrid formed in step (a) with the polypeptide according to the invention, DATP, dCTP, dGTP and dTTP, and an oligonucleotide or primer which is complementary to the first strand cDNA;
whereby dsDNA is obtained.

In another aspect, the present invention encompasses a method for determining the nucleotide base sequence of a DNA molecule, comprising the steps of:
(a) contacting said DNA molecule with a primer molecule able to hybridize to said DNA molecule;
(b) incubating said hybrid formed in step (a) in a vessel containing four different deoxynucleoside triphosphates, a DNA polymerase polypeptide according to the invention, and one or more DNA synthesis terminating agents which terminate DNA synthesis at a specific nucleotide base, wherein each said agent terminates DNA synthesis at a different nucleotide base; and
(c) separating the DNA products of the incubating reaction according to size, whereby at least a part of the nucleotide base sequence of said DNA can be determined.

In a preferred embodiment, said terminating agent is a dideoxynucleoside triphosphate.

A DNA synthesis terminating agent which terminates DNA synthesis at a specific nucleotide base refers to compounds, including but not limited to, dideoxynucleosides having a 2′,3′ dideoxy structure (e.g., ddATP, ddCTP, ddGTP and ddTTP). Any compound capable of specifically terminating a DNA sequencing reaction at a specific base may be employed as a DNA synthesis terminating agent.

In another aspect, the present invention encompasses a method for amplification of a DNA molecule comprising the steps of:
(a) incubating said DNA molecule in the presence of a polypeptide having DNA polymerase according to the invention, the terminal protein of the archaeal ampullavirus ABV and a mixture of different deoxynucleoside triphosphates.

In a preferred embodiment, the method for amplification of a DNA molecule according to the invention is characterized in that at one end of said DNA molecule a fragment containing the replication origin of said ABV is covalently bound.

Indeed, it is likely that ABV performs replication in a way similar to that of phi29, the sequences of the inverted terminal repeat (ITR) and the surrounding region should be involved in replication initiation. The sequences SEQ ID NO: 5 (left end) and SEQ ID NO: 6 (right end) are the sequences of both genomic termini including the ITR.

In a preferred embodiment, the sequence of the fragment containing the replication origin of said ABV comprises the sequences SEQ ID NO: 5 (left end) and SEQ ID NO: 6 (right end).

In a further aspect, the present invention is directed to a kit for sequencing a DNA molecule, comprising:
(a) a first container means comprising the polypeptide according to the invention;
(b) a second container means comprising one or more dideoxyribonucleoside triphosphates; and
(c) a third container means comprising one or more deoxyribonucleoside triphosphates.

The present invention also encompasses a kit for amplifying a DNA molecule, comprising:
(a) a first container means comprising the polypeptide according to the invention; and
(b) a second container means comprising one or more deoxyribonucleoside triphosphates.

In a more preferred embodiment, the kit for amplifying a DNA molecule according to the invention further comprises the isolated terminal protein of archaeal ampullavirus ABV corresponding to the polypeptide having the SEQ ID NO: 3 encoded by the ORF163 (SEQ ID NO: 4) of the ABV genome.

The present invention also comprises the use of a polypeptide according to the invention for implementing rolling circle amplification, multiple displacement amplification or protein-primed amplification method.

These particular methods are well known by the skilled person and are for example described in the documents:
Lizardi et al., 1998; Baner et al., 1998; Dean et al., Genome Res., 11, 1095-1099, 2001;
Larsson et al., Nature methods, 1, 227-232, 2004; for isothermal rolling-circle amplification method;
Dean et al., 2002, for multiple displacement amplification method; and
Blanco et al., 1994, for protein-primed amplification method.

In a preferred embodiment, the method according to the invention, the kit according to the invention or the use according to the invention is characterized in that the DNA polymerase polypeptide according to the invention is a polypeptide having DNA polymerase activity and deficient exonuclease activity (at least less than 1%, preferably less than 0.1% of the activity normally associated with the wild type ABV DNA polymerase).

The exonuclease activity associated with the DNA polymerase polypeptides of the invention can not significantly interfere with the use of the polymerase in a DNA sequencing, synthesizing or amplification reaction. However, it is preferred that the level of exonuclease activity be reduced to a level which is less than 10% or 1%, preferably less than 0.1% of the activity normally associated with DNA polymerases isolated from cells infected with the naturally-occuring ABV or having the sequence SEQ ID NO: 1.

The present invention is also directed to an apparatus for DNA sequencing or amplification having a reactor comprising a DNA polymerase polypeptide of the present invention.

The present invention also provides methods for producing anti-DNA polymerase polypeptide of the invention comprising, exposing an animal having immunocompetent cells to an immunogen comprising a polypeptide of the invention or at least an antigenic portion (determinant) of a polypeptide of the invention under conditions such that immunocompetent cells produce antibodies directed specifically against the polypeptide of the invention, or epitopic portion thereof. In one embodiment, the method further comprises the step of harvesting the antibodies. In an alternative embodiment, the method comprises the step of fusing the immunocompetent cells with an immortal cell line under conditions such that a hybridoma is produced.

Such antibodies can be used particularly for purifying the polypeptide of the present invention in a sample where others components are present.

The following examples and the figures are given for the purpose of illustrating various embodiments of the invention and are not meant to limit the present invention in any fashion.

### LEGENDS OF THE FIGURES

FIG. 1. Electron micrographs of particles of ABV after negative staining with 3% uranyl acetate. Bars, 100 nm.

FIG. 2. Estimation of the genome size by running intact (left panel) and restriction enzyme-digested viral DNA (right panel) in an agarose gel. Lane 1, intact viral DNA; lane 2 and 3, Age I and Afl II digested DNA respectively, M1, Lambda DNA-mono cut mix size marker from New England Biolabs (Catalog N3019S); M2, Ladder DNA size marker from Amersham.

FIG. 3. Genome map of ABV showing the location and size of the putative genes present on the two DNA strands. Most Genes are expressed on one strand as indicated by right-pointing arrows and a few on the complementary strand as shown by left-pointing arrows. Dark arrows indicate ORFs assigned functions while hypothetical genes are shown by gray arrows. Three internal ORFs are denoted by empty arrows and their sizes are in brackets. The map was drawn using MacPlasmap 2.05 and Adobe Illustrator.

FIG. 4. Sequence alignment between ORF653 (SEQ ID NO: 1) and the Phi29 DNA polymerase (issued from SEQ ID NO: 7 (GenBank Accession number 1XI1B) which corresponds to the DNA polymerase type-B family) showing two insertions (TPR I and II) which are specific for all known protein-priming DNA polymerase sequences. The conserved motifs involved in the exonuclease activity (Exo I, II, III) and in polymerisation (Pol I, IIa, IIb, III and IV) are indicated. Numbers indicate the amino acid lengths between the sequences.

FIGS. 5A and 5B:

FIG. 5A. Purification of recombinant polymerase encoded by ORF653 from E. coli. Lane 1, protein size marker; 2, total crude of the induced cells; 3, supernatant after sonication and centrifugation; 4, flow through after binding of the His-tagged protein to Ni-NTA agarose resin; 5, washed-out of the resin column; 6, purified protein. The size (kD) of polypeptides in the marker is shown at the left side. Two arrows indicate the position of the intact (upper) and fragmented (lower) polymerase.

FIG. 5B. Polymerization assay. The concentration of polymerase in the reaction was shown on top while the position of the 18-nt primer and the elongated molecules (42-nt) is indicated at the left side.

FIGS. 6A and 6B:

FIG. 6A. Secondary structure of the putative RNA element involved in ABV packaging.

FIG. 6B. Secondary structure of the prohead RNA of phi29. The seven helices conserved in the bacteriophage pRNAs are labelled by A to F from 5′ termini to 3′ termini while original designations was made according to the lengths of the stems (Bailey et al., 1990).

FIG. 7. Depiction of genomic content at the left end of ABV, phi29 and adenovirus (type 5). The length of ITR is 580, 6 and 103 bp repectively. Dark box denotes the region involved in packaging, where transcription direction is shown for ABV and ø29 by small arrows. Genes encoding polymerase (pol) and terminal protein (TP) are presented by light and dark gray arrows, respectively. Number of ORFs present between pol and the packing element is indicated in brackets.

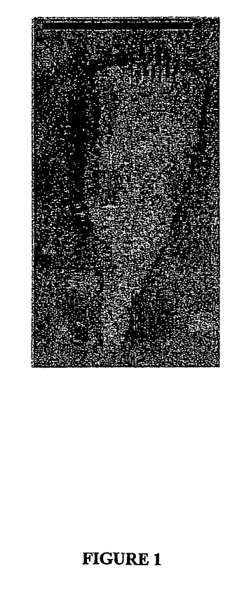

### EXAMPLE 1

### Materials and Methods

Nucleotides, primers and enzymes

(γ-³²P)ATP, dNTPs and enzymes were obtained from Pharmacia. Oligonucleotide polF1 (5′-CCTCCCTATTTGATAGGC-3′ SEQ ID NO: 8) was 5′-labeled with (γ-³²P)ATP and T4 polynucleotide kinase and electrophoretically purified on 8M urea-20% polyacrylamide gels. Labeled polF1 was mixed with polF1c+24 (5′-AGGTAAGCATGCATCAGTTAATACGCCTATCAAATAGGGAGG-3′ SEQ ID NO: 9) and the mixture was used as primer-template DNA molecule in the polymerization assay (see below).

### Purification of Viruses and Preparation of Viral DNA

Aerobic enrichment cultures were prepared from samples taken from a water reservoir in the crater of the Solfatara volcano at Pozzuoli, Italy, at 87-93° C. and pH 1.5-2, as described earlier (Häring et al., 2005a). They were grown at 75° C., pH 3. Virions were purified by centrifugation in a CsCl buoyant density gradient and disrupted with 1% (w/v) SDS for 1 hour at room temperature prior to extracting and precipitating DNA as described earlier (Häring et al., 2005a).

Sequencing of Genomic DNA

Given that a total of only about 200 ng purified viral DNA was available for the project, initially, about 1 ng DNA was amplified in vitro to yield a few μg using the GenomiPhi amplification kit (Amersham Biotech, Amersham). A shot-gun library was then constructed from sonicated DNA fragments in the size range 1.5 to 4 kbp, cloned into the SmaI site of pUC18. The library produced a highly biased genome coverage.

The assembly and sequence obtained from the amplified library was confirmed by preparing a mixed shotgun library using about 50 ng original ABV DNA and 1 μg DNA extracted from Acidianus betalipothrixviruses (Vestergaard et al., in prep.). The library was prepared as described above except that larger sonicated DNA fragments were cloned in the range 2 to 6.5 kbp. PCR reactions were performed to verify the regions where were not covered by the second library and a few clones were also sequenced further by primer walking (Peng et al., 2001).

### Sequence Analyses

BlastP search was performed against NCBI database. SMART and MotifScan were used to detect conserved domains, profiles or patterns. Coiled coils, secondary structures and transmembrane helices were detected by programs in ExPASy Proteomics Tools (http://www.expasy.org/tools/).

### Cloning and Purification of the Polymerase (ORF653)

ORF653 was PCR amplified from the original viral DNA using two primers containing NdeI and XhoI restriction sites, respectively (5′-TATTTTTACATATGCTACAAATCCT-3′ SEQ ID NO: 10 and 5′-TATAACTCGAGTGAGAGAATACTATTTAAGTC-3′ SEQ ID NO: 11). The PCR product was firstly cloned into pGEM-T vector (Promega) and the purified plasmid containing the ORF653 insert was digested with NdeI and XhoI. DNA fragment containing ORF653 with cohensive NdeI and XhoI ends was separated from pGEM-T fragment by low-melting agarose (Promega) gel electrophoresis and purified using gel extraction kit (Quiagen). The purified fragment was subsequently cloned into pET30-a vector (Novagen) digested with NdeI and XhoI and treated by calf intestinal phosphatase. The construct contains sequence encoding 6-histidine residues following the C-terminal end of the product of ORF653. Construct was sequenced to verify the sequence of the inserted ORF653 before transformation into the expression host cell Rosetta (Novagen).

A single colony of Rosetta transformant was inoculated into 5 ml LB medium containing 25 μg/ml Kanamycin and Chloramphenicol and incubated at 37° C. until OD reaches 0.5. The 5 ml culture was then transformed to 250 ml LB medium containing the same antibiotics. Cells were harvested after overnight growing at 30° C. in the presence of 0.1 mM IPTG and 1% ethanol. The his-tagged protein was purified using Ni-NTA His.Bind resins according to the protocol provided by the company (Novagen) and checked by SDS-PAGE.

### Polymerization Assay

The hybrid molecule polF1/polF1c+24 (described above) contains a 24-nucleotide long 5′-protuding end, and therefore can be used as primer-template for DNA polymerization. The reaction mixture contained, in 10 μl, 25 mM Tris-HCl (pH 7.6), 1 mM Dithiothreitol, 10 mM MgCl, 250 μM each of the four dNTPs, 0.1 μM of the primer-template DNA molecule and increasing concentration of recombinant polymerase. After incubation for 20 minutes at 50° C., the reaction was stopped by addition of 5 μl loading buffer (80% formamide, 10 mM EDTA, 50 μg/ml bromophenol blue) and heating for 3 minutes at 80° C. Samples were analyzed by 8M urea-20% PAGE and autoradiography. Polymerization was detected by as an increase in the size of the 5′-labeled primer strand (polF1).

### EXAMPLE 2

### Genome Sequence and Organisation

Nucleic acid was isolated from ABV virions and shown to be insensitive to RNase A but digestible by type II restriction endonucleases consistent with it being ds DNA. Given the low amount of genomic DNA that was available (<200 ng purified DNA), we adopted a two-step genome sequencing strategy.

First, about 1 ng DNA was amplified in vitro to yield about 2 μg DNA using the GenomiPhi amplification kit (Amersham Biotech). A shot-gun library was then constructed (see Materials and Methods) which produced a highly biased genome coverage, similar to that observed earlier for genomic DNA of the archaeal rudivirus SIRV1 which was amplified by the same procedure (Peng et al., 2004). A high level of chimeric clones were also produced. The genome was sequenced with, on average, a 20-fold coverage, and the sequences of the chimeric clones were identified by their lower frequency in the contigs and they were eliminated from the library.

In order to confirm the sequence assembly obtained from the amplified DNA library, about 50 ng of the original ABV DNA was mixed with 1 μg DNA extracted from Acidianus lipothrixviruses (Vestergaard et al., 2005) and a mixed shotgun library was prepared with larger cloned inserts (2 to 6.5 kbp). Sequencing and assembly of these ABV clones into those of the first library showed that the sequences from the two libraries matched exactly. Moreover, sequences of regions not covered by the second library were verified after PCR amplification of these regions, or primer walking, both performed on viral DNA and/or large insert clones. In addition, sequences of a few clones at the left terminus were extended by primer walking which yielded a final contig of about 22 kb.

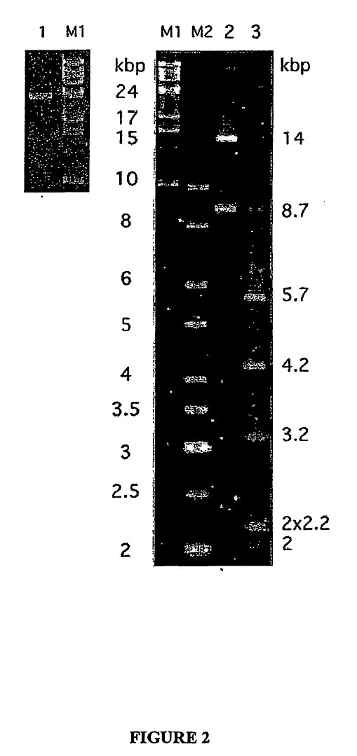

To confirm the genome assembly, about 40 ng of the viral DNA was digested with the restriction enzymes AgeI and AflII. The products were fractionated by agarose gel electrophoresis together with the intact viral DNA and the bands were stained with SYBR Gold (Invitrogen). Fragment sizes were consistent with the sequence of the assembled contig and the band of the intact viral DNA indicates a genome size of about 23.8 kb (FIG. 2).

The discrepancy between the size estimate from the restriction digests and the single contig size probably reflects that terminal regions of linear viral genomes are not represented in clone libraries (Häring et al., 2004; Häring et al., 2005b). Therefore, we attempted to sequence further out from the contig ends by primer-walking on amplified DNA. This yielded about 2 kb of additional sequence beyond which sequence reads invariably terminated. The total sequence obtained was 23,794 bp, consistent with the restriction fragment digest estimate. The G+C-content was 35%.

The genome exhibits inverted terminal repeats (ITRs) of 580 bp, smaller than those of the genomes of the rudiviruses (Peng et al., 2001) but similar to those of the archaeal betalipothrixviruses (Vestergaard et al., in prep.).

In order to test whether the genomes can circularise, PCR experiments were performed with a few different pairs of primers, annealing near each end of the genome but none of these produced an amplified product (data not shown). We infer, therefore, that the genome of ABV is linear.

### EXAMPLE 3

### Gene Content

The genome was annotated and start codons (88% AUG, 6% GUG and 6% UUG), TATA-like promoter motifs and/or Shine-Dalgarno motifs were assigned as described earlier (Bettstetter et al., 2003, supplementary Table 1S). A map for the whole viral genome containing 59 putative ORFs ranging in size from 37 to 653 amino acids is presented in FIG. 3. Three ORFs, 653, 103 and 257, contain internal start codons which are preceded by Shine-Dalgarno motifs. The internal ORFs were thus also assigned as putative genes (FIG. 3 and Table 1). All ORFs except one are located on one strand between position 8.5 kb and the right end. Of the remainder, all except 3 (ORF247, ORF53a and ORF156) are located on the other strand between the left end and position 8.5 kb (FIG. 3). About 49% of the ORFs shown in FIG. 3 are preceded by putative promoter sequences, and 68% are preceded by putative Shine-Dalgarno motifs. Moreover, about 11% of the ORFs exhibit downstream T-rich putative terminators. About 85% of the ORFs are arranged in putative operons and about 25% of the genes are predicted to generate transcripts that are either leaderless or carry very short leaders. The distance between ORFs is generally very short and 24% of the ORFs overlap with upstream ORF indicating that the genome is compact. Very strikingly, the 29 ORFs located between positions 10 kb to 21 kb appear to form one single big operon (FIG. 3 and Table 1S).

Only three ORFs could be assigned unambiguous functions based on homologue searches in public sequence databases (see Materials and Methods). ORF653 showed a significant sequence similarity with family B DNA polymerases with the best matches to protein-primed polymerases. Moreover, ORF156 was identified as a thymidylate kinase and ORF315 as a putative glycosyl transferase.

All the gene annotations are summarized in Table 1. While the majority of sequenced crenarchaeal viral genomes encode at least one ribbon-helix-helix (RHH) domain protein which is the most common gene product in crenarchaeal viruses, no RHH domain was detected in the genome of ABV. However, ORF56 shows a limited similarity to tetR-type helix-turn-helix domain which is present in some prokaryotic transcription regulators involved in resistance against drugs or stress. Another three ORFs contain leucine zipper pattern which may be involved in transcription regulation (FIG. 3 and Table 1). ORF188 contains a significant EF-hand calcium-binding domain and shows limited similarity to regulatory subunit of type II protein kinase A R-subunit. Therefore, it may encode a Ca⁺⁺ dependant protein kinase. A putative apaG domain profile was detected in the sequence of ORF133 which may be involved in protein-protein interactions. The secondary structure prediction of ORF133 protein sequence revealed about 90% extended strand and random coil. This correlates with the high content of beta-sheets in the tertiary structures of different apaG proteins. Three adjacent ORFs (112, 166 and 346) contain a few transmembrane helices and appear to be putative membrane or membrane-bound protein. Of special interest Is ORF346 which carry putative prokaryotic membrane lipoprotein lipid attachment site and EGF-like domain. The latter generally occur in the extracellular domain of membrane-bound proteins or in secreted proteins (Table 1). These properties are consistent with ORF346 constituting a viral coat protein which interacts with host membrane proteins, or a transmembrane protein which facilitates the release of viral particles from host cells. The putative transmembrane proteins encoded by the two upstream ORFs (112 and 166) might also be involved in the same process. The C-terminal sequences of both ORF346 and the downstream ORF470 show low complexity as observed in a few large ORFs in other crenarchaeal viral genomes (e.g. Häring et al., 2005; Neumann and Zillig, 1990). Function(s) of the proteins is unknown.

While three ORFs were assigned unambiguous function and a few carry putative conserved motifs or patterns (Table 1), the only gene shared between ABV and other crenarchaeal viruses is ORF315, the glycosyltransferase. Previously, comparative genomics revealed no or very few genes shared between different crenarchaeal viral genomes (Häring et al., 2004; Peng et al., 2001; Bettstetter et al., 2003). The result from this work reinforces that crenarchaeal viruses form an extremely diverse group.

TABLE 1
Functions assigned to ORFs of ABV

| | | Pfam entry | |
|---|---|---|---|
| | | or prosite | e- |
| ORF | Predicted function | document number | value* |
| 53a | Leucine zipper | Pdoc00029 | |
| 56 | HTH domain (TetR-type) | Pdoc00830 | 1.4 |
| 92a | Leucine zipper | Pdoc00029 | |
| 133 | ApaG domain profile, protein-protein | Pdoc51087 | 0.94 |
| | interaction | | |
| 150 | Leucine zipper | Pdoc00029 | |
| 188 | EF-Hand Ca⁺⁺ binding domain | Pdoc00018 | |
| | Regularoty subunit of type II protein | Pfam02197 | 0.0024 |
| | kinase A R-subunit | | |
| 346 | Lipoprotein-lipid attachment site | Pdoc00013 | |
| | EGF-like domain | Pdoc00021 | |
| 156 | Thymidylate kinase | Pfam02223 | 6.1e−5 |
| 315 | Glycosyl transferase | Pfam00534 | 3e−10 |
| 470 | Coiled coil, protein-protein inter- | | |
| | action | | |
| 653 | DNA polymerase | Pfam03175 | |

*e-value is not given to hits from Prosite pattern

### EXAMPLE 4

### DNA Replication

With the exception of the rudiviruses, we have little insight into the replication mechanisms of archaeal viral genomes. However, ABV is exceptional in that its genome encodes a putative protein-primed DNA polymerase. These enzymes are invariably encoded in linear ds DNA genomes carrying ITRs with covalently linked terminal proteins and have been characterised in both a bacteriophage, ø29, and in a eukaryal adenovirus (reviewed by Salas, 1991). The replication initiation model for these viruses involves a free terminal protein forming a heterodimer with the DNA polymerase and interacting with the replication origin via the viral DNA-bound terminal protein and specific nucleotide sequences at either end of the genome. A hydroxyl group of serine, threonine or tyrosine in the terminal protein serves as the recipient site for the first nucleotide. Moreover, many linear ds DNA plasmids and mitochondrial genomes exhibit ITRs and protein-priming DNA polymerases and carry terminal proteins which are likely to replicate in a similar way (Salas, 1991). The subfamily of protein-priming DNA polymerases belongs to the DNA-dependent DNA polymerase family B and possesses two insertions, TPR-1 and TPR-2 (Blasco et al., 2000; Dufour et al., 2000; Rodriguez et al., 2005).

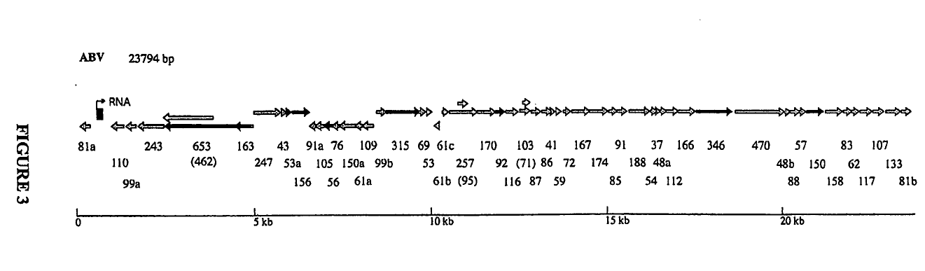

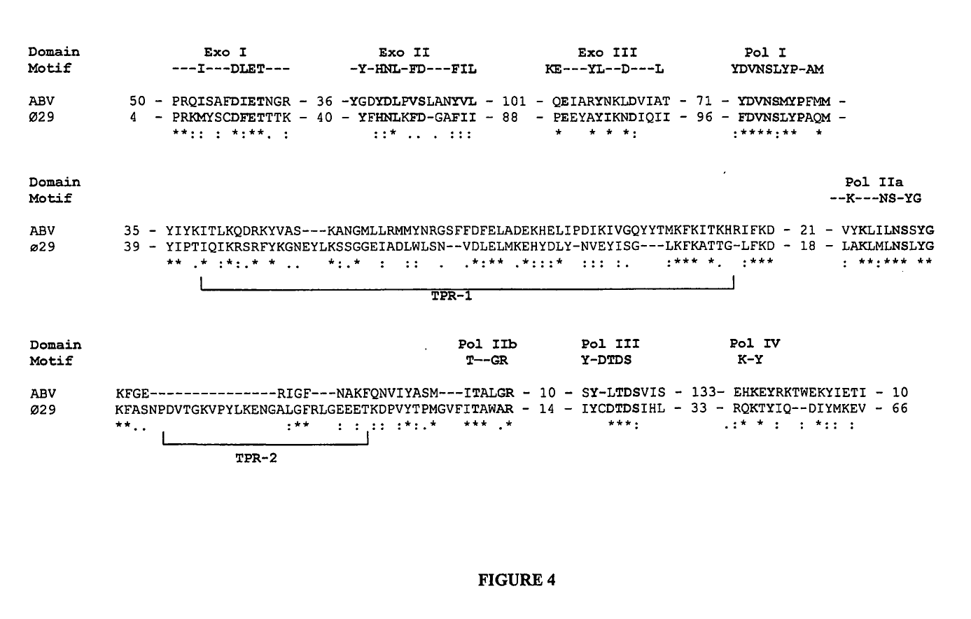

A sequence alignment of ORF653 and the ø29 DNA polymerase (FIG. 4) illustrates that ORF653 contains three exonuclease domains (Exo I, II and III) in the N-terminal region and five conserved synthetic domains (pol I, Ia, IIb, III and IV) in the C-terminal part characteristic of family B DNA polymerases (Blanco et al., 1991; Rohe et al., 1992). Moreover, the insertions TPR-1 and TPR-2 are also present in ORF653 (FIG. 4). While TPR-1 is similar in size (50 aa) to those of all the protein-priming DNA polymerases, including that encoded by human adenovirus (Dufour et al., 2000), whereas TPR-2, located between motifs pol IIa and IIb, is truncated relative to the known size range of inserts extending from 28 aa (ø29) to 118 aa (adenovirus type 2) (Bois et al., 1999). For the ø29 DNA polymerase, TPR-1 was found to participate in the interaction with the terminal priming protein (Dufour et al., 2000) while TPR-2 was shown to be required for the high processivity and strand-displacement activity of the polymerase (Rodriguez et al., 2005). Although the function of the more conserved TPR-1 is likely to be general for all protein-priming DNA polymerases, it remains unclear whether this applies to the more variable TPR-2.

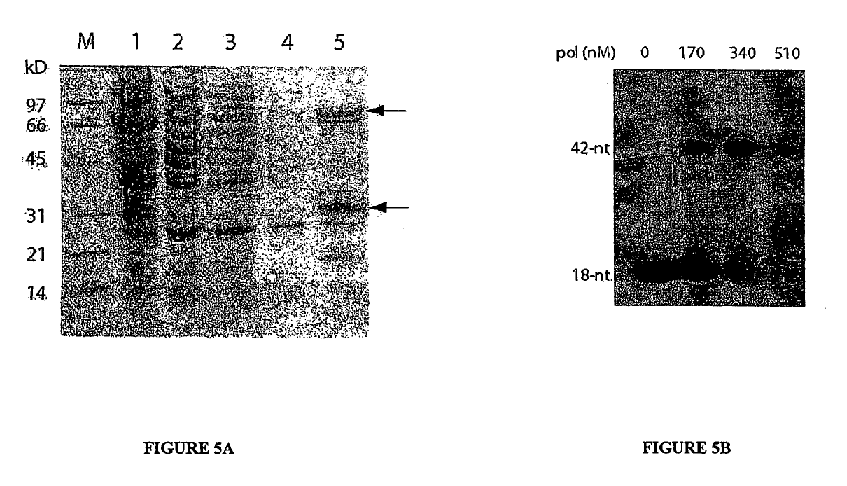

In order to confirm that ORF653 is a DNA polymerase, the gene was amplified from the viral genome by PCR and cloned into an E. coli expression vector. A reasonable amount of soluble recombinant protein was purified together with a C-terminal fragment of the protein (FIG. 5A). To test the polymerization activity, the protein was incubated with a labelled primer-template DNA at 50° C. FIG. 5B clearly shows that the primer (18 nt) was elongated to the size of the template (42 nt) indicative of polymerization activity. When NTPs was used instead of dNTPs, no polymerization was detected (data not shown). This confirms that the product of ORF653 is indeed a DNA polymerase.

### EXAMPLE 5

### Terminal Protein

The presence of an ITR and a gene encoding a putative protein-priming DNA polymerase in the linear genome of ABV strongly suggests that each 5′ terminus is covalently attached to a terminal protein. This is difficult to test experimentally, owing to the very low yields of virus particles that are produced. Therefore, we analyzed terminal protein sequences from relevant bacteriophages, linear plasmids and human adenoviruses in order to gain insights into conserved features of these proteins. While the polymerase is relatively conserved, the terminal protein shows very low conservation. For example, the terminal protein of E. coli bacteriophage PRD1 shows no significant sequence similarity with other known terminal proteins (Savilahti et al., 1987) and only 13/48% identity/similarity was found between the terminal protein of ø29 and a linear mitochondrial plasmid of white-rot fungus Pleurotus ostreatus (Kim et al., 2000). However, the gene location of the terminal protein is highly conserved. Thus, in bacteriophages ø29, PRD1, GA-1 and CP-1 (Accession numbers P03681, P09009, X96987 and Z47794) and in human adenoviruses the gene is always located immediately upstream of the DNA polymerase gene whereas the size of the protein ranges between 230 aa and 266 aa for the bacteriophages and 671 aa for adenovirus type 2 (AC_{—}000007). DNA replication was less studied for the linear plasmids, two of which were found to encode a fused N-terminal terminal protein and a C-terminal DNA polymerase (Kim et al., 2000; Takeda et al., 1996). Sequence alignment of the DNA polymerases also revealed large sequence extensions at the N-terminal part in the other linear plasmids (Bois et al., 1999), indicating that the genes of the polymerase and terminal protein may generally be fused. Moreover, transcript mapping revealed that the DNA polymerase and terminal protein genes are always cotranscribed into a single mRNA in all the studied viruses, including CP-1 (Martin et al., 1996) and ø29 family phages (reviewed by Meijer et al., 2001). Thus, the polymerase and terminal protein are closely linked in both gene organization and function. For ABV, the gene upstream of the polymerase encodes 163 aa which is two thirds the size of the bacteriophage terminal proteins. However, sequence alignments using ClustalW (EMBL-EBI) revealed higher score between ORF163 and TP from PRD1 than between TPs of PRD1 and ø29 (data not shown), indicating that ORF163 may encode a terminal protein.

### EXAMPLE 6

### Viral DNA Packaging

Earlier, it was shown that the virion structure of ABV is very complex when compared with other known crenarchaeal viruses. The bottle-shaped virion contains a “stopper” at the narrow end, and a disk, or ring, bearing 20 short filaments at the broad end (Häring et al., 2005). The main body appears to be built up of two layers encasing a complex core and the nucleoprotein filament is packed, compactly, within the main body. Thus, the DNA packaging mechanism is likely to be complex.

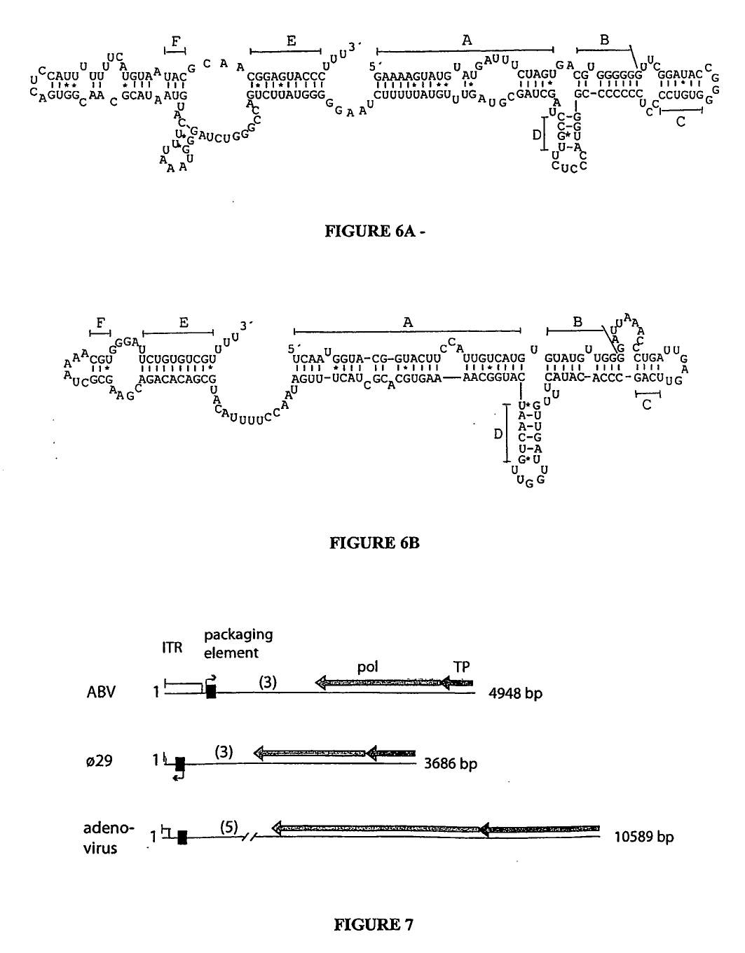

Packaging of genomic DNA has been studied for diverse bacteriophages and eukaryal viruses carrying linear genomes. The mechanisms share some common features, including the involvement of a pair of noncapsid proteins and the energy source, ATP, to translocate the long DNA molecule into a preformed procapsid (reviewed by Guo, 2005). An essential component of the ø29 packaging machinery is a 174-nt RNA, pRNA, which participates actively in DNA translocation by binding to the procapsid and ATP and cooperating with the packaging protein (Guo, 2005). The pRNA is encoded adjacent to the ITR at one end of ø29 genome and it exhibits a high level of secondary structure and conserved secondary structural motifs can form for all the known ø29 related bacteriophages (reviewed by Meijer et al., 2001). Moreover, a corresponding region, adjacent to an ITR, was also found to be important for the packaging of adenoviral DNA (Grable and Hearing, 1990). Examination of the corresponding regions in the ABV genome, revealed a 600-bp region, lacking open reading frames, close to the left ITR, which was relatively G+C-rich in the centre. The predicted secondary structure for the 200 bp G+C-rich sequence shows high similarity to that of pRNA from bacteriophages ø29 and CP-1 (FIG. 6). The seven helices labeled A to F are highly conserved in all pRNAs of bacteriophages. Differences occur only in the region to the left of helix F where extra hairpin-loops occur in the putative ABV RNA while a small loop is present in the bacteriophage pRNAs (FIG. 6). Transcription of the ABV RNA could be initiated at the promoter-like sequence, ATTTAAT, located 20 bp upstream of the element. The conserved genomic position, similar secondary structure, high G+C content and presence of a putative promoter all strongly indicate that this non-coding region encodes a RNA molecule which is probably involved in viral DNA packaging.

Another important component involved in ø29 DNA packaging is the connector which was proposed to rotate in order to translocate the DNA into the prohead (Meijer et al., 2001). Although the general morphology of ABV is different from that of ø29, the “stopper” resembles the connector of ø29 which also has a bottle-neck shape and the wide end of which is also buried inside the prohead (Meijer et al., 2001). Moreover, the broad end of the stopper is connected to the nucleoprotein filament (Häring et al., 2005). Therefore, the connector may also be involved in packaging of ABV.

Currently, little is known about the packaging of archaeal virions with linear DNA. One tends to speculate that it is simpler for the crenarchaeal rudiviruses and filamentous viruses which generally have the supercoiled genomic DNA arranged in long and “linear” structures (rod or filamentous shape) containing 1 to 3 proteins. Therefore, they seem not to pack the genomic DNA into a preformed structure. For viruses as ABV and PSV, which have more compact structure and especially lengthy linear genomes, one would infer that they need a comprehensive encapsidation or packaging mechanism.

Genomic content at the left end of ABV, bacteriophage ø29 and eukaryotic adenovirus is depicted in FIG. 7 which shows high similarity between the three viruses. ABV is the first archaeal virus which is reported to contain a protein-primed DNA polymerase. The presence of the polymerase in three morphologically distinct viruses from three domains of life strongly indicates the protein-primed DNA replication mechanism is ancient, probably existed prior to the divergence of three domains of life.

### REFERENCES

Altschul, S. F., T. L. Madden, A. A. Schäffer, J. Zhang, Z. Zhang, W. Miller, and D. J. Lipman. 1997. Gapped BLAST and PSI-BLAST: a new generation of protein database search programs. Nucl. Acids Res. 25:3389-3402.
Arnold, H. P., W. Zillig, U. Ziese, I. Holz, M. Crosby, T. Utterback, J. F. Weidmann, J. Kristjansson, H. P. Klenk, K. E. Nelson, and C. M. Fraser. 2000. A novel lipothrixvirus, SIFV, of the extremely thermophilic crenarchaeon Sulfolobus. Virology 267:252-266.
Barthelemy, I., M. Salas, and R. P. Mellado. 1986. In vivo transcription of bacteriophage ø29 DNA: transcription initiation sites. J. Virol. 60:874-879.
Bettstetter, M., X. Peng, R. A. Garrett, and D. Prangishvili. 2003. AFV1, a novel virus infecting hyperthermophilic archaea of the genus Acidianus. Virology 315:68-79.
Blum, H., W. Zillig, S. Mallock, H. Domday, and D. Prangishvili. 2001. The genome of the archaeal virus SIRV1 has features in common with genomes of eukaryal viruses. Virology 281:6-9.
Blanco L, Lazaro J M, de Vega M, Bonnin A, Salas M. Terminal protein-primed DNA amplification. Proc Natl Acad Sci USA. Dec. 6, 1994;91(25):12198-202.
Bois F, Barroso G, Gonzalez P, Labarere J. 1999. Molecular cloning, sequence and expression of Aa-polB, a mitochondrial gene encoding a family B DNA polymerase from the edible basidiomycete Agrocybe aegerita. Mol. Gen. Genet. 261:508-513.
Bravo, A., B. Illana, and M. Salas. 2000. Compartmentalization of phage ø29 DNA replication: interaction between the primer terminal protein and the membrane-associated protein p1. EMBO J. 19:5575-5584.
Brügger, K., P. Redder, and M. Skovgaard. 2003. MUTAGEN: Multi User Tool for Annotating GENomes. Bioinformatics 19:2480-2481.
Dufour, E, J. Mendez, J. M. Lazaro, M. de Vega, L. Blanco and M. Salas. 2000. An aspartic acid residue in TPR-1, a specific region of protein-priming DNA polymerases, is required for the functional interaction with primer terminal protein. J. Mol. Biol. 304:289-300.
Guo P. 2005. Bacterial virus phi29 DNA-packaging motor and its potential applications in gene therapy and nanotechnology. Methods Mol Biol. 300:285-324.
Grable M, Hearing P. 1990. Adenovirus type 5 packaging domain is composed of a repeated element that is functionally redundant. J. Virol. 64:2047-56.
Hatfield, L., and P. Hearing. 1993. The NFIII/OCT-1 binding site stimulates adenovirus DNA replication in vivo and is functionally redundant with adjacent sequences. J. Virol. 67:3931-3939.
Häring, M., X. Peng, K. Brügger, R. Rachel, K. O. Stetter, R. A. Garrett, and D. Prangishvili. 2004. Morphology and genome organization of the virus PSV of the hyperthermophilic archaeal genera Pyrobaculum and Thermoproteus: a novel virus family, the Globuloviridae. Virology 323:233-242.
Häring, M., R. Rachel, X. Peng, R. A. Garrett, and D. Prangishvili 2005a. Diverse viruses in hot springs of Pozzuoli, Italy, including a unique bottle-shaped archaeal virus ABV from a new family, the Ampullaviridae. J. Virol. 79:9904-9911.
Häring, M., Vestergaard, G., Brügger, K., Rachel, R., Garrett, R. A. and Prangishvili, D. 2005b. Structure and genome organisation of AFV2, a novel filamentous archaeal virus with unusual terminal structures. J. Bacteriol. 187:3855-3858.
Häring, M. et al., 2005c. Nature in press.
Janekovic, D., S. Wunderl, I. Holz, W. Zillig, A. Gierl, and H. Neumann. 1983. TTV1, TTV2 and TTV3, a family of viruses of the extremely thermophilic, anaerobic sulfur reducing archaebacterium Thermoproteus tenax. Mol. Gen. Genet. 192:39-45.
Kim, E. K., J. H. Jeong., H. S. Youn, Y. B. Koo and J. H. Roe. 2000. The terminal protein of a linear mitochondrial plasmid is encoded in the N-terminus of the DNA polymerase gene in white-rot fungus Pleurotus ostreatus. Curr. Genet. 38:283-290.
Martin, A. C., R. Lopez and P. Garcia. 1996. Analysis of the complete nucleotide sequence and functional organization of the genome of Streptococcus pneumoniae bacteriophage Cp-1. J Virol. 70:3678-3687.
Meijer, W. J., J. A. Horcajadas and M. Salas. 2001. Phi29 family of phages. Microbiol. Mol. Biol. Rev. 65:261-287.
Peng, X., H. Blum, Q. She, S. Mallok, K. Brügger, R. A. Garrett, W. Zillig, and D. Prangishvili. 2001. Sequences and replication of genomes of the archaeal rudiviruses SIRV1 and SIRV2: Relationships to the archaeal lipothrixvirus SIFV and some eukaryal viruses. Virology 291: 226-234.
Peng, X., A. Kessler, H. Phan, R. A. Garrett, and D. Prangishvili. 2004. Multiple variants of the archaeal DNA rudivirus SIRV1 in a single host and a novel mechanism of genome variation. Mol. Microbiol. 54:366-375.
Picardeau, M., J. R. Lobry, and B. J. Hinnebusch. 1999. Physical mapping of an origin of bidirectional replication at the centre of the Borrelia burgdorferi linear chromosome. Mol. Microbiol. 32:437-445.
Prangishvili, D., K. M. Stedman, and W. Zillig. 2001. Viruses of the extremely thermophilic archaeon Sulfolobus. Trends Microbiol. 9:39-42.
Prangishvili, D. and R. A. Garrett. 2004. Exceptionally diverse morphotypes and genomes of crenarchaeal hyperthermophilic viruses. Biochem. Soc. Trans. 32:204-208.
Prangishvili, D. and Garrett R. A., 2005. Viruses of hyperthermophilic Crenarchaea. Trends Microbiol. 13:535-542.
Rachel, R., M. Bettstetter, B. P. Hedlund, M. Häring, A. Kessler, K. O. Stetter, and D. Prangishvili. 2002. Remarkable morphological diversity of viruses and virus-like particles in terrestrial hot environments. Arch. Virol. 147:2419-2429.
Rodriguez, I., J. M. Lazaro, L. Blanco, S. Kamtekar, A. J. Berman, J. Wang, T. A. Steitz, M. Salas, and M. de Vega. 2005. A specific subdomain in phi29 DNA polymerase confers both processivity and strand-displacement capacity. Proc. Natl. Acad. Sci. USA. 102:6407-12.
Salas, M. 1991. Protein-priming of DNA replication. Annu. Rev. Biochem. 60:39-71.
Savilahti H. and D. H. Bamford. 1987. The complete nucleotide sequence of the left very early region of Escherichia coli bacteriophage PRD1 coding for the terminal protein and the DNA polymerase. Gene 57:121-130.
Takeda M., H. Hiraishi, T. Takesako, S. Tanase and N. Gunge. 1996. The terminal protein of the linear DNA plasmid pGKL2 shares an N-terminal domain of the plasmid-encoded DNA polymerase. Yeast 12:241-246.
Torarinsson, E., H.-P. Klenk, and R. A. Garrett. 2005. Divergent transcriptional and translational signals in Archaea. Environ. Microbiol. 7:47-54.
Vestergaard, G., M. Haering, X. Peng, R. Rachel, R. A. Garrett, and D. Prangishvili. 2005. ARV1, a rudivirus infecting the hyperthermophilic archaeal genus Acidianus. Virology, 336:83-92.
Zillig, W., D. Prangishvili, C. Schleper, M. Elferink, I. Holz, S. Albers, D. Janekovic, and D. Götz. 1996. Viruses, plasmids and other genetic elements of thermophilic and hyperthermophilic Archaea. FEMS Microbiol. Rev. 18:225-236.

1. An isolated DNA polymerase selected from the group of polypeptides consisting of:
a) the polypeptide having the amino acid sequence of SEQ ID NO: 1;
b) a fragment of a) having a DNA polymerase activity;
c) a polypeptide comprising at least the SEQ ID NO: 1 fragments allowing the DNA polymerase activity of said DNA polymerase of a);
d) a polypeptide having the amino acid sequence of SEQ ID NO: 1 wherein the exonuclease sites Exo I, Exo II and/or Exo III as identified in FIG. 4 have been mutated or deleted in order that the resulting DNA polymerase polypeptide has significantly less or no detectable exonuclease activity compared to the polypeptide having the amino acid sequence of SEQ ID NO: 1;
e) a polypeptide having sequence which is at least 80% identity after optimum alignment with the sequence SEQ ID NO: 1, said polypeptide having a DNA polymerase activity.

2. The DNA polymerase of claim 1, which is isolated from the Archaeal Ampullavirus ABV.

3. The DNA polymerase of claim 1, which comprises at least the Pol I, Pol IIa, Pol IIb, Pol III and Pol IV fragments of SEQ ID NO: 1 as identified in FIG. 4.

4. A nucleic acid encoding a DNA polymerase polypeptide according to claim 1.

5. A vector comprising the nucleic acid of claim 4.

6. The vector of claim 5, wherein said nucleic acid is operably linked to a promoter.

7. The vector of claim 5, which has been deposited at the C.N.C.M. (Collection Nationale de Cultures de Microorganismes, Institut Pasteur, Paris, France) the 28 Apr. 2006 under the number I-3601.

8. A host cell comprising the vector of claim 5.

9. The host cell of claim 8, which has been deposited at the C.N.C.M. (Collection Nationale de Cultures de Microorganismes, Institut Pasteur, Paris, France) the 28 Apr. 2006 under the number I-3601.

10. A method of producing a DNA polymerase, said method comprising:
(a) culturing the host cell of claim 8 in conditions suitable for the expression of said nucleic acid; and
(b) isolating said DNA polymerase from said host cell.

11. The method of claim 10, wherein said host cell is a prokaryotic or an eukaryotic cell.

12. A method of synthesizing a double-stranded DNA molecule comprising:
(a) hybridizing a primer to a first DNA molecule; and
(b) incubating said DNA molecule of step (a) in the presence of one or more deoxyribonucleoside triphosphates or analogs thereof and the polypeptide of claim 1, under conditions sufficient to synthesize a second DNA molecule complementary to all or a portion of said first DNA molecule.

13. A method of synthesizing a single-stranded DNA molecule comprising:
(a) the synthesis of a double-stranded DNA molecule by a method according to claim 12; and
(b) denaturing the double-stranded DNA molecule obtained in step (a); and
(c) recovering the single-stranded DNA molecule obtained in step (b).

14. A method for production of DNA molecules of greater than 10 kilobases in length comprising the methods of claim 12, wherein the first DNA molecule:
which serve as a template in step (a) is greater than 10 kilobases.

15. The method of claim 12, wherein said deoxyribonucleoside triphosphates are selected from the group consisting of dATP, dCTP, dGTP and dTTP.

16. A method for amplifying a double stranded DNA molecule, comprising:
(a) providing a first and second primer, wherein said first primer is complementary to a sequence at or near the 3′-termini of the first strand of said DNA molecule and said second primer is complementary to a sequence at or near the 3′-termini of the second strand of said DNA molecule;
(b) hybridizing said first primer to said first strand and said second primer to said second strand in the presence of the polypeptide of claim 1, under conditions such that a nucleic acid complementary to said first strand and a nucleic acid complementary to said second strand are synthesized;
(c) denaturing
said first and its complementary strands; and
said second and its complementary strands; and
(d) repeating steps (a) to (c) one or more times.

17. A method of preparing cDNA from mRNA, comprising:
(a) contacting mRNA with an oligo(dT) primer or other complementary primer to form a hybrid, and
(b) contacting said hybrid formed in step (a) with the DNA polymerase of claim 1 and dATP, dCTP, dGTP and dTTP, whereby a cDNA-RNA hybrid is obtained.

18. A method of preparing dsDNA from mRNA, comprising:
(a) contacting mRNA with an oligo (dT) primer or other complementary primer to form a hybrid; and
(b) contacting said hybrid formed in step (a) with the polypeptide of claim 1, dATP, dCTP, dGTP and dTTP, and an oligo nucleotide or primer which is complementary to the first strand cDNA;
whereby dsDNA is obtained.

19. A method for determining the nucleotide base sequence of a DNA molecule, comprising the steps of:
(a) contacting said DNA molecule with a primer molecule able to hybridize to said DNA molecule;
(b) incubating said hybrid formed in step (a) in a vessel containing four different deoxynucleoside triphosphates, a DNA polymerase polypeptide of claim 1, and one or more DNA synthesis terminating agents which terminate DNA synthesis at a specific nucleotide base, wherein each said agent terminates DNA synthesis at a different nucleotide base; and
(c) separating the DNA products of the incubating reaction according to size, whereby at least a part of the nucleotide base sequence of said DNA can be determined.

20. The method of claim 19, wherein said terminating agent is a dideoxynucleoside triphosphate.

21. A method for amplification of a DNA molecule comprising the steps of:
(a) incubating said DNA molecule in the presence of a polypeptide having DNA polymerase of claim 1, the terminal protein of the archaeal ampullavirus ABV and a mixture of different deoxynucleoside triphosphates.

22. A method for amplification of a DNA molecule according to claim 21, wherein at one end of said DNA molecule a fragment containing the replication origin of said ABV is covalently bound.

23. A kit for sequencing a DNA molecule, comprising:
(a) a first container means comprising the polypeptide of claim 1;
(b) a second container means comprising one or more dideoxyribonucleoside triphosphates; and
(c) a third container means comprising one or more deoxyribonucleoside triphosphates.

24. A kit for amplifying a DNA molecule, comprising:
(a) a first container means comprising the polypeptide of claim 1; and
(b) a second container means comprising one or more deoxyribonucleoside triphosphates.

25. A kit of claims 24, further comprising an isolated or recombinant terminal protein of archaeal ampullavirus ABV having the sequence SEQ ID NO: 3.

26. Use of a polypeptide of claim 1 for rolling circle amplification, multiple displacement amplification or protein-primed amplification.

27. The method of claim 10, wherein said polypeptide as defined in d) having deficient exonuclease activity and a DNA polymerase activity.

28. An apparatus for DNA sequencing or amplification having a reactor comprising a DNA polymerase polypeptide of claim 1.

29. The kit of claim 23, wherein said polypeptide as defined in d) having deficient exonuclease activity and a DNA polymerase activity.

30. The use of claim 26, wherein said polypeptide as defined in d) having deficient exonuclease activity and a DNA polymerase activity.
